(19) United States
(12) Patent Application Publication
(10) Pub. No.: US 2011/0183598 A1
(43) Pub. Date: Jul. 28, 2011

(54) Method and System for Controlling Microbiological Contamination in Buildings

(76) Inventor: Alton R. Holt, Silsbee, TX (US)

(21) Appl. No.: 12/693,918

(22) Filed: Jan. 26, 2010

(51) Int. Cl. F24F 7/007; A61L 9/015; A61L 9/18; B01J 19/08; B01J 21/06

(52) U.S. Cl. 454/338; 422/121; 422/4

(57) ABSTRACT

A method and system control microbes in buildings. In one embodiment, the system includes controlling microbes in a building using an air conditioning system. The air conditioning system takes air and conditions air to be introduced to the building. The system also includes a purification device disposed in the air conditioning system. The purification device includes a catalyst comprising titanium dioxide and a light disposed to emit electromagnetic radiation into the catalyst. The purification device is disposed to allow the air to flow into the purification device and contact the catalyst with a reaction producing hydrogen peroxide gas. The system also includes air conditioning ductwork in the building. Treated air including the hydrogen peroxide gas flows from the air conditioning system to the ductwork. The system also includes a vent disposed in a room of the building. The treated air flows through the ductwork to the vent and in to the room. The hydrogen peroxide gas controls microbes in the room by degrading the microbes.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

This invention relates to the field of antimicrobials and more specifically to the field of controlling microbial contamination in buildings using hydrogen peroxide gas as an antimicrobial.

2. Background of the Invention

There is an increasing need for disinfection and protection of buildings and rooms from bacteria, viruses, mold, and the like. Conventional disinfection processes involve the application of detergents and liquid sanitizers. Drawbacks to such conventional methods include inefficiencies disinfecting in certain locations such as between walls. Further drawbacks include inefficiencies in the frequency of the disinfection. For instance, such conventional disinfection processes are typically carried out on a daily basis or intermittently during a day. Developments over such conventional processes include using hydrogen peroxide as a disinfectant. Disinfectant processes using hydrogen peroxide include vaporizing liquid hydrogen peroxide solutions to create a mist of water droplets containing hydrogen peroxide. Drawbacks include that such hydrogen peroxide mist may not be used in occupied spaces because the mist typically contains hundreds to thousands of parts per million of hydrogen peroxide. Further drawbacks include inefficiencies in disinfecting a volume of space because the droplets in the mist precipitate out of the air. Additional drawbacks include that the hydrogen peroxide in the mist is surrounded by water, which may insulate the hydrogen peroxide molecules in the droplets and may prevent the molecules from being drawn to the microbes in the air or on surfaces by electrostatic attraction.

Protection of buildings has experienced an increasing need in light of recent activities such as terrorist activities. However, drawbacks to protection of buildings and providing safe areas in the buildings against terrorist attacks has involved evacuation of the buildings to protect and remove any microbiological contaminants.

Consequently, there is a need for an improved antimicrobial system for protecting and removing microbiological contamination of facilities. Further needs include providing safe areas within buildings against microbiological contamination.

### BRIEF SUMMARY OF SOME OF THE PREFERRED EMBODIMENTS

These and other needs in the art are addressed in one embodiment by a system for controlling microbes in a building. The system includes an air conditioning system. The air conditioning system takes air and conditions treated air to be introduced to the building. The system also includes a purification device disposed in the air conditioning system. The purification device includes a catalyst comprising titanium dioxide and a light disposed to emit electromagnetic radiation into the catalyst. The purification device is disposed to allow the air to flow into the purification device and contact the catalyst with a reaction producing hydrogen peroxide gas. The system also includes air conditioning ductwork in the building. The treated air including the hydrogen peroxide gas flows from the air conditioning system to the ductwork. The system also includes a vent disposed in a room of the building. The treated air flows through the ductwork to the vent and into the room. The hydrogen peroxide gas controls microbes in the room by degrading the microbes.

These and other needs in the art are addressed in another embodiment by a system for preventing microbes from entering a room. The system includes a blower that blows air to a purification device. The system also includes the purification device having a catalyst comprising titanium dioxide and a light disposed to emit electromagnetic radiation into the catalyst. The purification device is disposed to allow the air to flow into the purification device and contact the catalyst, with a reaction producing hydrogen peroxide gas. The system further includes treated air including the hydrogen peroxide gas flowing from the purification device to a wall space of the room. The treated air is at a pressure higher than the pressure of the wall space.

These and other needs in the art are addressed in a further embodiment by a method for controlling microbes in a building. The method includes treating air in a purification device to produce hydrogen peroxide gas. The purification device is disposed in an air conditioning system that takes in the air. The purification device includes a catalyst having titanium dioxide and a light disposed to emit electromagnetic radiation into the catalyst. The purification device is disposed to allow the air to flow into the purification device and contact the catalyst, with a reaction producing the hydrogen peroxide gas. The treated air including the hydrogen peroxide gas exits the air conditioning system. The method also includes introducing the treated air to the building. The method further includes controlling the microbes in the building with the hydrogen peroxide gas.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other embodiments for carrying out the same purposes of the present invention. It should also be realized by those skilled in the art that such equivalent embodiments do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 illustrates an embodiment of a microbe control system for a building;

FIG. 2 illustrates an embodiment of a purification device;

FIG. 3 illustrates an embodiment of a catalyst;

FIG. 4 illustrates an embodiment of a microbe control system for a protected room;

FIG. 5 illustrates an embodiment of a purification device; and

FIG. 6 illustrates an embodiment of a microbe control system having a purification device for a building and a purification device for a protected room.

### DETAILED DESCRIPTION OF THE PREFERRED EMBODIMENTS

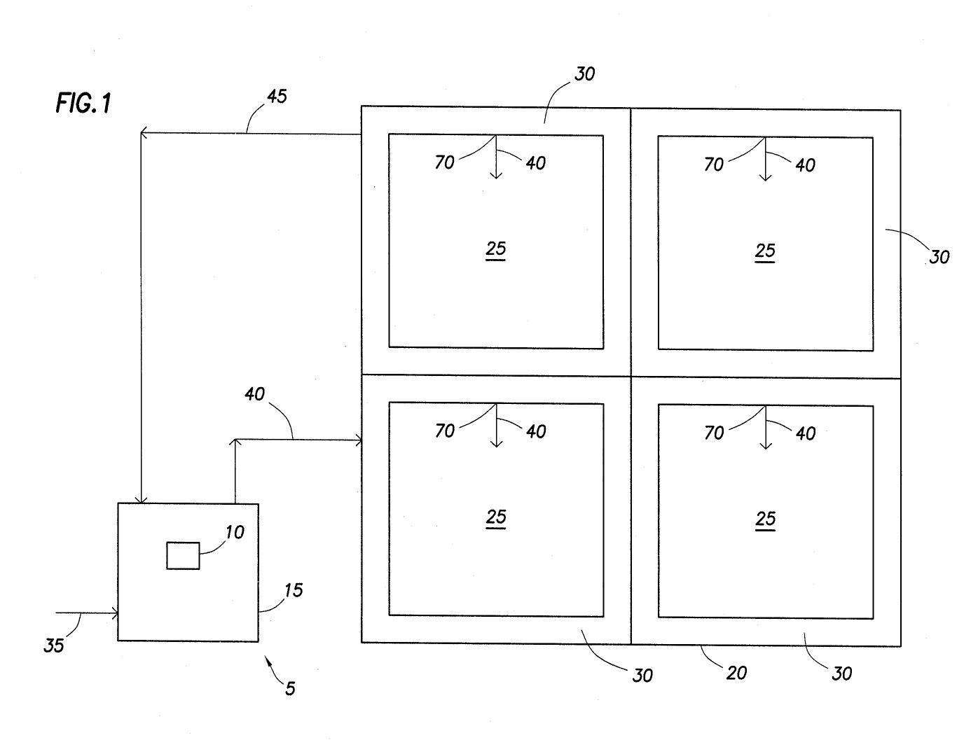

FIG. 1 illustrates an embodiment of a microbe control system 5 for a building 20. Microbe control system 5 creates hydrogen peroxide gas and supplies the hydrogen peroxide gas to building 20 to control microbiological contaminants in building 20. The microbiological contaminants include any type of microbe. In an embodiment, the microbes comprise fungi, mold, viruses, bacteria, or any combinations thereof. Microbe control system 5 controls the microbiological contaminants by degrading all or a portion of the microbiological contaminants in building 20 (i.e., by killing the microbiological contaminants).

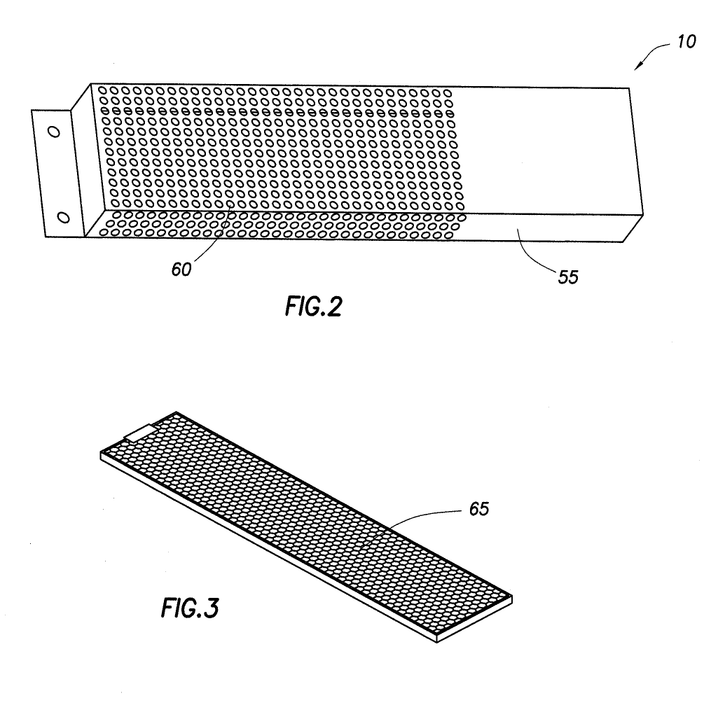

In an embodiment as illustrated in FIG. 1, microbe control system 5 includes purification device 10 disposed within air conditioning system 15. Purification devices are disclosed in U.S. application Ser. No. ______, filed on ______, 2010 and entitled “Microbe Reduction and Purification” with attorney docket number 1820-00100. U.S. application Ser. No. ______ is hereby incorporated by reference in its entirety. FIG. 2 illustrates an embodiment of purification device 10 having body 55, device openings 60, and catalyst 65 (not illustrated). Device openings 60 allow air in air conditioning system 15 to flow into purification device 10 and contact catalyst 65, which is disposed within the interior of purification device 10. Purification device 10 may include any suitable number of catalysts 65. FIG. 3 illustrates an embodiment of catalyst 65. Catalyst 65 comprises titanium dioxide. In other embodiments, catalyst 65 comprises titanium dioxide and metallic additives. Any metallic additives suitable for improving the reaction to produce the hydrogen peroxide gas may be used. In an embodiment, the metallic additives include copper, silver, rhodium, or any combinations thereof. Catalyst 65 may have any suitable configuration for use in purification device 10. In embodiments, catalyst 65 comprises a configuration of a plurality of cells. In an embodiment as illustrated in FIG. 2, catalyst 65 comprises a configuration of a plurality of hexagonal, walled cells (i.e., honeycomb shape configuration). Without being limited by theory, the hexagonal, walled cell configuration facilitates the reaction to produce the hydrogen peroxide because it provides an increased surface area for the reaction. The embodiment of catalyst 65 shown in FIG. 2 has a rectangular shape, but it is to be understood that catalyst 65 is not limited to a rectangular shape. In alternative embodiments, catalyst 65 may have any other suitable shape such as a square shape, triangular shape, and the like. In some embodiments (not illustrated), catalyst 65 is disposed inside purification device 10 at an angle in relation to the direction at which the air flows into purification device 10 and contacts catalyst 65. In embodiments, catalyst 65 is disposed at angle in relation to the direction at which the air flows into purification device 10 and contacts catalyst 65 with the angle between about 15 degrees and about 75 degrees, and alternatively at about 45 degrees. Without being limited by theory, disposing catalyst 65 at an angle to the direction at which the air flows into purification device 10 increases the surface area available for the reaction to produce the hydrogen peroxide. For instance, as light and air pass through catalyst 65, the catalyst 65 disposed at an angle increases the amount of contact of the light and air with the surface of catalyst 65. Catalyst 65 includes a light (not illustrated) disposed inside the catalyst 65. The light is a non-ozone producing ultraviolet light. In embodiments, the light is a crystal ultraviolet light. Without limitation, commercial examples of non-ozone producing ultraviolet lights include the non ozone bulb provided by LightTech. Catalyst 65 includes one light. In alternative embodiments, catalyst 65 includes more than one light. The light is disposed to emit electromagnetic radiation into catalyst 65. For instance, the light emits electromagnetic radiation into the hexagonal, walled cells of catalyst 65 with the electromagnetic radiation contacting the surface of the cells.

As shown in FIG. 1, air conditioning system 15 is any type of air conditioning system suitable for allowing purification device 10 to be disposed and operate within and also for providing air conditioned air to a building.

FIG. 1 illustrates an embodiment of operation of microbe control system 5 with a building 20 having rooms 25 with microbe control system 5 controlling microbiological contaminants in building 20. In an embodiment as illustrated, air conditioning system 15 takes in air 35 and conditions air 35 (i.e., cools the air). In embodiments, air 35 is ambient air. Purification device 10 may be disposed in air conditioning system 15 at any location suitable for producing hydrogen peroxide gas from air 35 and providing the hydrogen peroxide gas in treated air 40. In some embodiments, purification device 10 is disposed in air conditioning system 15 after air 35 has been exposed to the coils (not illustrated). In other embodiments, purification device 10 is disposed in air conditioning system 15 before air 35 has been exposed to the coils. In such embodiments, the produced hydrogen peroxide gas controls microbes on the coils. Without limitation, when the microbes grow on the coils, the microbes provide insulation on the coils, which requires additional energy to operate air conditioning system 15. The hydrogen peroxide gas thereby improves the efficiency of air conditioning system 15 in such embodiments. In alternative embodiments (not illustrated), microbe control system 5 has a purification device 10 disposed after air 35 has been exposed to the coils and another purification device 10 disposed before air 35 has been exposed to the coils. Air 35 flows into purification device 10 and contacts catalyst 65 with the air passing through catalyst 65. It is to be understood that the ambient air has a moisture content and is comprised of water vapor and oxygen. Catalyst 65 and the moisture in the ambient air (i.e., the water vapor and oxygen) are exposed to the electromagnetic radiation from the lights. A reaction between the titanium dioxide, the moisture in the ambient air, and the electromagnetic radiation produce the hydrogen peroxide gas. In an embodiment, the reaction is a photo-catalytic reaction. For instance, in embodiments, moisture from the ambient air contacts catalyst 65 as it flows through catalyst 65. The electromagnetic radiation from the light contacts the various surfaces of catalyst 65 and reacts with the moisture against the titanium dioxide to produce the hydrogen peroxide gas. The reaction in purification device 10 to produce the hydrogen peroxide gas does not produce ozone.

Without being limited by theory, the produced hydrogen peroxide gas has both positive and negative charges. With such charges, the hydrogen peroxide gas is drawn to microbes by electrostatic attraction. For instance, the hydrogen peroxide gas is drawn to the positive and negative charges of the surface of the microbes. The hydrogen peroxide gas then controls the microbes by chemically degrading the microbes, which may be degraded cell by cell. In embodiments in which the microbes are attached to structures such as a wall, the hydrogen peroxide gas degrades the microbes down to the point of attachment. In some instances, the microbes release from the surface and may be removed. In embodiments, the microbes may be removed without removing structurally sound material. The hydrogen peroxide gas also diffuses into porous material (i.e., anywhere that air flows) such as porous walls and cloth, which allows degradation of the microbes behind the walls or in the cloth.

As illustrated in FIG. 1, treated air 40 includes the conditioned air from air conditioning system 15 and the hydrogen peroxide gas produced from purification device 10. Treated air 40 is fed to the air conditioning ductwork (not illustrated) of building 20. Treated air 40 flows through the air conditioning ductwork to each room 25, and treated air 40 flows into each room 25 through the vents 70 of each room 25. The hydrogen peroxide gas in treated air 40 controls microbes in rooms 25. For instance, the hydrogen peroxide gas controls the microbes on the walls, furniture, clothing, and in the air of rooms 25. The hydrogen peroxide gas in treated air 40 also controls the microbes in the air conditioning ductwork. In embodiments, return air 45 from building 20 flows back through the ductwork to air conditioning system 15 and is conditioned along with air 35.

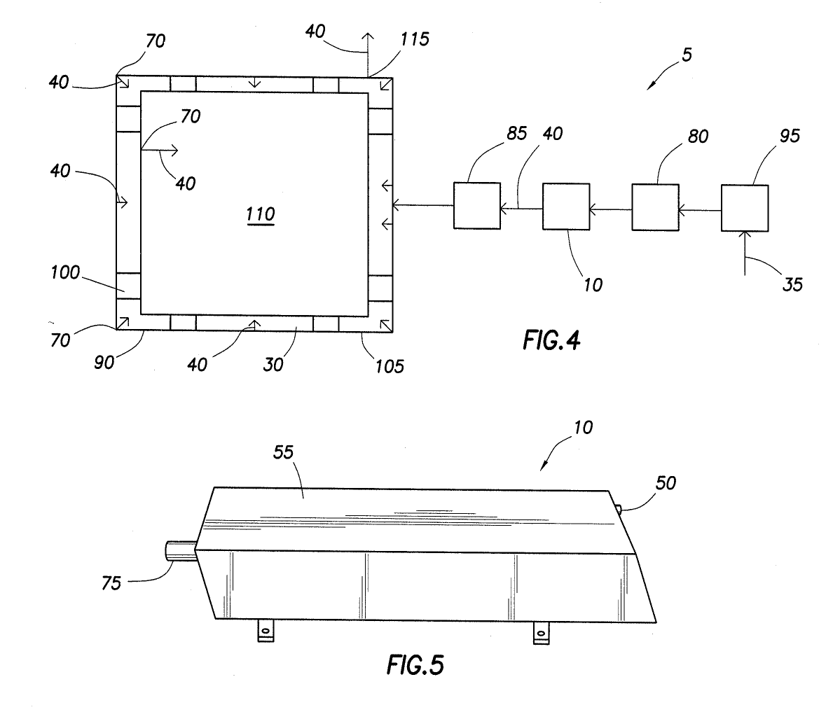

FIG. 4 illustrates an embodiment of microbe control system 5 in which treated air 40 controls microbes in a protected room 90. As shown, blower 95 takes air 35 and blows air 35 into filter 80. Blower 95 may be any blower suitable for taking ambient air and blowing the air through a filter. In an embodiment, blower 95 produces sufficient pressure on air 35 to pass air through filter 80, purification device 10, absorber 85, and to provide a desired pressure in wall space 30 of protected room 90. Filter 80 may be any filter suitable for removing particulates from air. Without limitation, examples of suitable filters 80 include high efficiency particulate air filters (HEPA filters), ultra low particulate air filters (ULPA filters), and the like. In an embodiment, filter 80 removes particulates from air 35 to facilitate cleaning of catalyst 65 (i.e., keeping catalyst 65 clear of such particulates). Pressure from blower 95 passes air 35 through filter 80 to purification device 10. The filtered air 35 flows into purification device 10 and contacts catalyst 65, and the reaction of catalyst 65, light, and air 35 produce the hydrogen peroxide gas. It is to be understood that purification device 10 may have different configurations depending on the type of use. One of the embodiments of purification device 10 suitable for use in the embodiment of microbe control system 5 illustrated in FIG. 4 is shown in FIG. 5. In such an embodiment, the filtered air 35 enters purification device 10 at gas inlet 50, which is on a longitudinal end of purification device 10. In such an embodiment, air 35 flows into purification device 10 and contacts catalyst 65 inside body 55, producing the hydrogen peroxide gas. Treated air 40 containing air and the hydrogen peroxide gas exit purification device 10 through gas outlet 75.

In some embodiments as illustrated in FIG. 4, treated air 40 is fed to absorber 85. Absorber 85 removes further particulates from treated air 40. In some embodiments, absorber 85 is a nuclear grade activated carbon filter. In such embodiments, the hydrogen peroxide gas in treated air 40 controls the microbes on the carbon in the nuclear grade activated carbon filter. Without limitation, controlling such microbes improves the efficiency of the nuclear grade activated carbon filter because, otherwise, the microbes may begin to fill up the filter and reduce its absorption capability. Treated air 40 flows through absorber 85 to wall space 30 of protected room 90. Treated air 40 flows into wall space 30 of protected room 90 by vents 70. In embodiments, treated air 40 is at a pressure greater than that of wall space 30 and interior 110. In some embodiments, treated air 40 flows to each vent 70 by ductwork or the like. Wall space 30 refers to the interior space of wall 105 of protected room 90. The hydrogen peroxide gas in treated air 40 controls microbes in wall space 30. In some embodiments, a vent 70 is disposed between each wall stud 100. Without limitation, each wall stud 100 then provides its own self-sealing area. By feeding the pressurized treated air 40 into wall space 30, microbe control system 5 provides a positive pressure in wall space 30. Such positive pressure facilitates prevention of microbes from accessing protected room 90 when access (i.e., doors) is closed. For instance, if a breach occurs in wall 105 from outside of protected room 90, pressure of the pressurized wall space 30 fills the breach with treated air 40, with treated air 40 flowing out through the breach. The hydrogen peroxide gas in treated air 40 controls any microbes at the breach, and the flow of treated air 40 then carries any microbes out of the breach and away from protected room 90. With any breach of wall 105 from interior 110 of protected room 90, treated air 40 flows into interior 110 through the breach. In some embodiments, protected room 90 has an exhaust 115 by which treated air 40 flows out of wall 105. In other embodiments, treated air 40 also flows into interior 110 through vents 70.

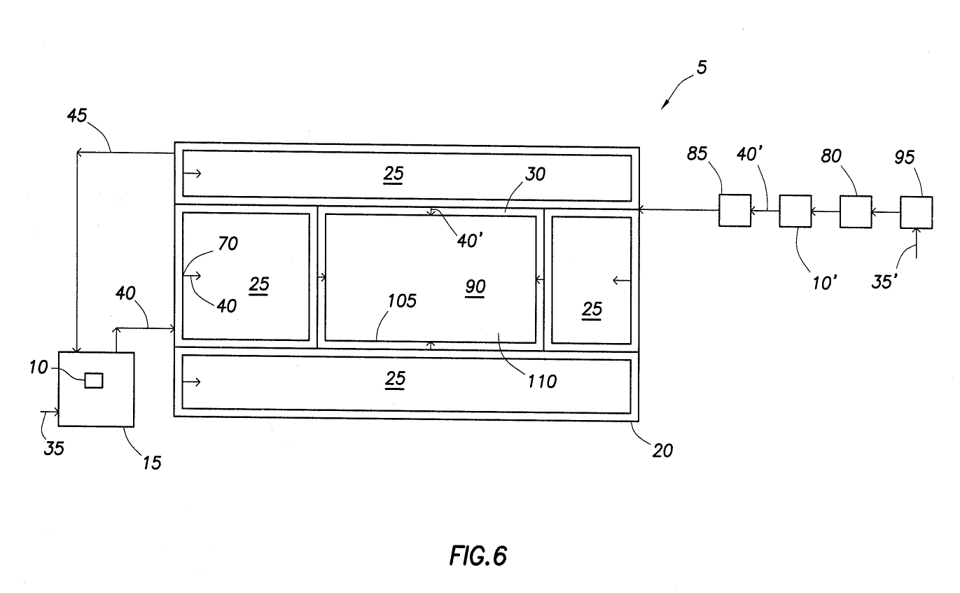

FIG. 6 illustrates an embodiment of microbe control system 5 having a purification device 10 disposed in air conditioning system 15 that provides treated air 40 to rooms 25 of building 20 and also purification device 10′ that provides treated air 40′ to protected room 90 in building 20. In such embodiment, protected room 90 is disposed within building 20. In an embodiment as illustrated, protected room 90 is an interior room of building 20 with no windows to the exterior of building 20. In some embodiments as illustrated, protected room 90 has rooms 25 on each side of protected room 90. In operation of the embodiment illustrated in FIG. 6, purification device 10 produces hydrogen peroxide gas from air 35, which flows from air conditioning system 15 to the ductwork of building 20 in treated air 40. From the ductwork, treated air 40 flows into rooms 25 through vents 70. Microbes in the ductwork and also in rooms 25 are controlled by the hydrogen peroxide gas in treated air 40. In an embodiment in which a breach of building 20 occurs, the hydrogen peroxide gas in building 20 may attack the microbes entering building 20 at the breach, which allows any occupants of building 20 to escape to protected room 90. For instance, if a bomb containing microbes such as chemical or biological warfare agents detonates near building 20 and shatters windows in one or more of rooms 25, the hydrogen peroxide gas in treated air 40 near the shattered windows attack the microbes entering the room through the shattered windows. In embodiments, newly produced hydrogen peroxide gas is continuously fed to rooms 25 during and after the breach of building 20. With the hydrogen peroxide gas attacking the microbes near the breach (i.e., shattered windows), occupants of building 20 may move to protected room 90 and close any doors accessing protected room 90. In embodiments, blower 95 and purification device 10′ are then activated. Air 35′ is blown by blower 95 through filter 80 and to purification device 10′, which produces hydrogen peroxide gas. Treated air 40′ containing the produced hydrogen peroxide gas flows through absorber 85 to wall space 30 of protected room 90, pressurizing wall space 30. In embodiments, filter 80, hydrogen peroxide gas in treated air 40, and absorber 85 combine to remove particulates and/or control microbes in air 35′ prior to being fed to wall space 30. The pressurized wall space 30 containing the hydrogen peroxide gas prevents microbes (i.e., chemical warfare agents) from entering wall space 30 and also prevents the microbes from entering the wall space 30 and interior 110 if a breach of wall 105 occurs. In other embodiments, purification device 10′ is continuously running before a breach of building 20 occurs.

As illustrated, purification device 10′ may be disposed outside of building 20. In alternative embodiments (not illustrated), purification device 10′ may be disposed at any suitable location inside building 20.

Although the present invention and its advantages have been described in detail, it should be understood that various changes, substitutions and alterations may be made herein without departing from the spirit and scope of the invention as defined by the appended claims.

What is claimed is:

1. A system for controlling microbes in a building, comprising:
an air conditioning system, wherein the air conditioning system conditions air to be introduced to the building;
a purification device disposed in the air conditioning system, wherein the purification device comprises a catalyst comprising titanium dioxide and a light disposed to emit electromagnetic radiation into the catalyst, and wherein the purification device is disposed to allow the air to flow into the purification device and contact the catalyst, with a reaction producing hydrogen peroxide gas;
air conditioning ductwork in the building, wherein treated air comprising the hydrogen peroxide gas flows from the air conditioning system to the ductwork; and
a vent disposed in a room of the building, wherein the treated air flows through the ductwork to the vent and into the room, wherein the hydrogen peroxide gas controls microbes in the room by degrading the microbes.

2. The system of claim 1, wherein the catalyst comprises a plurality of hexagonal, walled cells.

3. The system of claim 1, wherein the light comprises a non-ozone producing ultraviolet light.

4. The system of claim 1, wherein the catalyst is disposed to provide a reaction surface by which the air reacts when exposed to the catalyst and the electromagnetic radiation.

5. The system of claim 1, further comprising a second purification device that produces hydrogen peroxide gas from a titanium dioxide catalyst.

6. The system of claim 5, wherein the hydrogen peroxide gas from the second purification device is fed to wall space of a protected room disposed in the building, and wherein the hydrogen peroxide gas is fed into the wall space at a pressure greater than pressure inside the wall space.

7. A system for preventing microbes from entering a room, comprising:
a blower that blows air to a purification device;
the purification device comprising a catalyst, wherein the purification device comprises titanium dioxide and a light disposed to emit electromagnetic radiation into the catalyst, and wherein the purification device is disposed to allow the air to flow into the purification device and contact the catalyst, with a reaction producing hydrogen peroxide gas; and
wherein treated air comprising the hydrogen peroxide gas flows from the purification device to a wall space of the room, and wherein the treated air is at a pressure higher than pressure of the wall space.

8. The system of claim 7, wherein the air passes through a filter before flowing into the purification device.

9. The system of claim 7, wherein the treated air flows through an absorber before flowing to the wall space.

10. The system of claim 7, wherein the light comprises a non-ozone producing ultraviolet light.

11. A method for controlling microbes in a building, comprising:
(A) treating air in a purification device to produce hydrogen peroxide gas, wherein the purification device is disposed in an air conditioning system that takes in the air, and wherein the purification device comprises a catalyst comprising titanium dioxide and a light disposed to emit electromagnetic radiation into the catalyst, and further wherein the purification device is disposed to allow the air to flow into the purification device and contact the catalyst, with a reaction producing the hydrogen peroxide gas, and wherein treated air comprising the hydrogen peroxide gas exits the air conditioning system;
(B) introducing the treated air to the building; and
(C) controlling the microbes in the building with the hydrogen peroxide gas.

12. The method of claim 11, wherein the catalyst comprises a plurality of hexagonal, walled cells.

13. The method of claim 11, wherein the light comprises a non-ozone producing ultraviolet light.

14. The method of claim 11, wherein the catalyst is disposed to provide a reaction surface by which the air reacts when exposed to the catalyst and the electromagnetic radiation.

15. The method of claim 11, wherein introducing the treated air to the building comprises the air flowing through the air conditioning ductwork of the building.

16. The method of claim 11, further comprising:
(D) treating air in a second purification device to produce hydrogen peroxide gas, wherein the second purification device comprises a titanium dioxide catalyst and a second light disposed to emit electromagnetic radiation into the titanium dioxide catalyst, and further wherein the second purification device is disposed to allow the air to flow into the second purification device and contact the catalyst, with a reaction producing the hydrogen peroxide gas; and
(E) introducing the hydrogen peroxide gas from the second purification device to a wall space of a room of the building.

17. The method of claim 16, further comprising pressurizing the wall space to a pressure greater than pressure inside of the room and pressure proximate to outside of the room.

18. The method of claim 16, further comprising feeding the air to second purification device with a blower.

19. The method of claim 16, further comprising filtering the air before the air flows into the second purification device.

20. The method of claim 16, further comprising absorbing particulates from air comprising the hydrogen peroxide gas before introducing the air to the wall space.
